(19) United States
(12) Patent Application Publication
(10) Pub. No.: US 2009/0036554 A1
(43) Pub. Date: Feb. 5, 2009

(54) OPHTHALMIC COMPOSITIONS COMPRISING A TERPENE COMPOUND

(76) Inventors: Susan E. Burke, Batavia, NY (US); Catherine Scheuer, West Henrietta, NY (US); Kimberly Millard, Rochester, NY (US)

Correspondence Address: Bausch & Lomb Incorporated, One Bausch & Lomb Place, Rochester, NY, 14604-2701, US

(21) Appl. No.: 12/177,171

(22) Filed: Jul. 22, 2008

Related U.S. Application Data
(60) Provisional application No. 60/953,219, filed on Aug. 1, 2007.

(51) Int. Cl. A61K 47/36; A61P 27/02

(52) U.S. Cl. 514/779

(57) ABSTRACT

An aqueous ophthalmic composition comprising one or more terpene compounds, and one or more natural polymers selected from the group comprising hyaluronic acid, condroitin sulfate, alginate, guar gum, fructan, arabinogalactan or any corresponding salt or derivative of each thereof. The compositions can be used to disinfect or package a contact lens, or as an eye drop to comfort irritated eyes or rewet a contact lens.

This application claims the benefit under 35 U.S.C. § 119(e) of U.S. provisional application Ser. No. 60/953,219 filed Aug. 1, 2007.

The invention relates to a contact lens composition comprising a terpene and a natural polymer, and the use of the composition to disinfect or package a contact lens, or as an eye drop to comfort irritated eyes or rewet a contact lens.

### BACKGROUND OF THE INVENTION

Various antimicrobial agents are known for use as disinfectants or preservatives in ophthalmic compositions such as contact lens care solutions or eye drops, particularly eye drops formulated for use with contact lenses. The antimicrobial agents should have a broad spectrum of antimicrobial activity and be non-irritating to the eye. Some of the most common antimicrobial agents used in ophthalmic applications include chlorhexidine, polyquaternium-1, poly(hexamethylene biguanide) and alexidine.

Each antimicrobial compound has its own degree of efficacy against a specific collection of microorganisms. Because a single antimicrobial compound may not be efficacious against all microorganisms of interest in a safe and effective concentration range, it is sometimes beneficial to introduce another antimicrobial compound into the formulation. The difficulty, however, arises in the appropriate selection of the two or more antimicrobial compounds because of unknown chemical interactions that can exist between the two compounds or with other formulation components, e.g., a potential interaction with a surfactant, or a comfort or wetting polymer such as many natural polymers.

U.S. Pat. No. 5,696,171 describes a composition for disinfecting contact lenses containing one or more terpene compounds. The composition can also include at least one agent selected from the group consisting of an oxidative system and an enzyme and optionally at least one other disinfecting agent such as a biguanide or polymeric quaternary ammonium compound.

The development of disinfecting ophthalmic compositions that are simple to use, effective against a broad spectrum of microorganisms and are non-toxic and do not cause ocular irritation is of great interest.

### SUMMARY OF THE INVENTION

The invention is directed to an aqueous ophthalmic composition comprising one or more natural polymers and one or more terpene compounds. The natural polymers are selected from the group comprising hyaluronic acid, condroitin sulfate, alginate, hydroxypropyl guar and arabinogalactan or any corresponding salt or derivative of each thereof. The ophthalmic composition also has an osmolality in a range from 200 mOsmol/kg to 400 mOsmol/kg.

The invention is also directed to the use of the compositions to disinfect or package a contact lens, or as an eye drop to comfort irritated eyes or rewet a contact lens.

### DETAILED DESCRIPTION OF THE INVENTION

The invention is directed to an aqueous ophthalmic composition comprising a terpene compound, and one or more natural polymers selected from the group comprising hyaluronic acid, condroitin sulfate, alginate, guar gum, fructan and arabinogalactan or any corresponding salt or derivative of each thereof. The ophthalmic composition will have an osmolality in a range from 200 mOsmol/kg to 400 mOsmol/kg. As used herein, the term “ophthalmic composition” denotes a composition intended for application in the eye or intended for treating a device to be placed in contact with the eye such as a contact lens.

Suitable terpene compounds for use in the ophthalmic compositions include any monoterpene, sesquiterpene and/or diterpene or derivatives thereof. Acyclic, monocyclic and/or bicyclic mono-, sesqui- and/or diterpenes, and those with higher numbers of rings, can be used. A “derivative” of a terpene as used herein shall be understood to mean a terpene hydrocarbon having one or more functional groups such as terpene alcohols, terpene ethers, terpene esters, terpene aldehydes, terpene ketones and the like and combinations thereof. Here, both the trans and also the cis isomers are suitable. The terpenes as well as the terpene moiety in the derivative can contain from 6 to about 100 carbon atoms and preferably from about 10 to about 25 carbon atoms.

Representative examples of suitable terpene alcohol compounds include verbenol, eugenol, transpinocarveol, cis-2-pinanol, nopol, isoborneol, carbeol, piperitol, thymol, α-terpineol, terpinen-4-ol, menthol, 1,8-terpin, dihydro-terpineol, nerol, geraniol, linalool, citronellol, hydroxycitronellol, 3,7-dimethyl octanol, dihydro-myrcenol, tetrahydro-alloocimenol, perillalcohol, falcarindiol and any one mixture thereof.

Representative examples of suitable terpene ether and terpene ester compounds include 1,8-cineole, 1,4-cineole, isobornyl methylether, rose pyran, α-terpinyl methyl ether, menthofuran, trans-anethole, methyl chavicol, allocimene diepoxide, limonene mono-epoxide, isobornyl acetate, nonyl acetate, α-terpinyl acetate, linalyl acetate, geranyl acetate, citronellyl acetate, dihydro-terpinyl acetate, meryl acetate and any one mixture thereof.

Representative examples of terpene aldehyde and terpene ketone compounds include myrtenal, campholenic aldehyde, perillaldehyde, citronellal, citral, hydroxy citronellal, camphor, verbenone, carvenone, dihydro-carvone, carvone, piperitone, menthone, geranyl acetone, pseudo-ionone, α-ionine, iso-pseudo-methyl ionone, n-pseudo-methyl ionone, iso-methyl ionone, n-methyl ionone and any one mixture thereof. Any other terpene hydrocarbons having functional groups known in the art can be used in the compositions.

In one embodiment, suitable terpenes or derivatives thereof as antimicrobial agents include, but are not limited to, tricyclene, α-pinene, terpinolene, carveol, amyl alcohol, nerol, β-santalol, citral, pinene, nerol, b-ionone, caryophillen (from cloves), guaiol, anisaldehyde, cedrol, linalool, d-limonene (orange oil, lemon oil), longifolene, anisyl alcohol, patchouli alcohol, α-cadinene, 1,8-cineole, ρ-cymene, 3-carene, ρ-8-mentane, trans-menthone, borneol, α-fenchol, isoamyl acetate, terpin, cinnamic aldehyde, ionone, geraniol (from roses and other flowers), myrcene (from bayberry wax, oil of bay and verbena), nerol, citronellol, carvacrol, eugenol, carvone, α-terpineol, anethole, camphor, menthol, limonene, nerolidol, farnesol, phytol, carotene (vitamin A₁), squalene, thymol, tocotrienol, perillyl alcohol, borneol, simene, carene, terpenene, linalool, 1-terpene-4-ol, zingiberene (from ginger) and any one mixture thereof. An exemplary list of the preferred terpene compounds includes α-terpineol, 1-terpinen-4-ol, eugenol, menthol and geraniol, with 1-terpinen-4-ol being the most preferred.

As stated, the ophthalmic compositions also include one or more natural polymers selected from the group consisting of hyaluronic acid, condroitin sulfate, alginate, hydroxylpropyl guar and arabinogalactan. The concentration of the natural polymers in the compositions is from 0.01% w/v to 0.5% w/v or from 0.05% w/v to 0.2% w/v.

A mixture of hyaluronic acid and alginate can also be used. The concentration of hyaluronic acid or salt thereof in the composition is from 0.01% w/v to 0.5% w/v or from 0.05% w/v to 0.2% w/v. The average molecular weight of the hyaluronic acid or salt thereof is from 500 kD to 5000 kD, or from 1000 kD to 3000 kD. The concentration of alginate in the composition is from 0.01% w/v to 0.5% w/v or from 0.05% w/v to 0.2% w/v. In one embodiment, the average molecular weight of the alginate is from 50 kD to 3,000 kD or from 200 kD to 2000 kD.

In another embodiment, a mixture of hyaluronic acid or salt thereof, or alginate, and hydroxypropylmethyl cellulose is present in the composition. The concentration of hydroxypropylmethyl cellulose is from 0.05% w/v to 3% w/v. The average molecular weight of the hydroxypropylmethyl cellulose is from 20 kD to 120 kD.

Hyaluronic acid is a linear polysaccharide (long-chain biological polymer) formed by repeating disaccharide units consisting of D-glucuronic acid and N-acetyl-D-glucosamine linked by β(1-3) and β(1-4) glycosidic linkages. Hyaluronic acid is distinguished from the other glycosaminoglycans, as it is free from covalent links to protein and sulphonic groups. Hyaluronic acid is ubiquitous in animals, with the highest concentration found in soft connective tissue. It plays an important role for both mechanical and transport purposes in the body; e.g., it gives elasticity to the joints and rigidity to the vertebrate disks, and it is also an important component of the vitreous body of the eye.

Hyaluronic acid is accepted by the ophthalmic community as a compound that can protect biological tissues or cells from compressive forces. Accordingly, hyaluronic acid has been proposed as one component of a viscoelastic ophthalmic composition for cataract surgery. The viscoelastic properties of hyaluronic acid, that is, hard elastic under static conditions though less viscous under small shear forces enables hyaluronic acid to basically function as a shock absorber for cells and tissues. Hyaluronic acid also has a relatively large capacity to absorb and hold water. The stated properties of hyaluronic acid are dependent on the molecular weight, the solution concentration, and physiological pH. At low concentrations, the individual chains entangle and form a continuous network in solution, which gives the system interesting properties, such as pronounced viscoelasticity and pseudoplasticity that is unique for a water-soluble polymer at low concentration.

Hyaluronic acid can be prepared by fermentation of bacteria such as streptococci. The bacteria are incubated in a sugar rich broth, and the produced hyaluronic acid is separated from impurities and purified. The molecular weight of hyaluronic acid produced via fermentation can be set by the sugars placed in the fermentation broth. Hyaluronic acid produced via fermentation is commercially available.

Alginate is an anionic biopolymers produced by a variety of microorganisms and marine algae. Alginate is a polysaccharide that comprises β-D-mannuronic acid units and (α-L-guluronic acid units. Some alginate polymers are block copolymers with blocks of the guluronic acid (or salt) units alternating with blocks of the mannuronic acid (or salt) units as depicted in-part below.

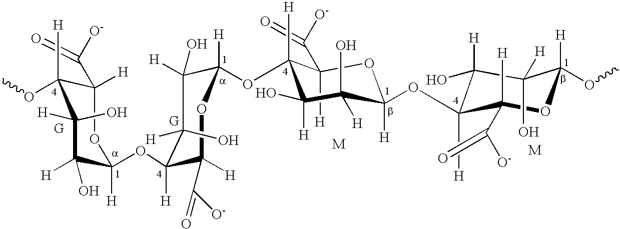

Some alginate molecules have single units of guluronic acid (or salt) alternating with single units of mannuronic acid (or salt). The ratio and distribution of the mannuronic and guluronic unit, along with the average molecular weight, affect the physical and chemical properties of the copolymer. See Haug, A. et al., Acta Chem. Scand., 183-90 (1966). Alginate polymers have viscoelastic rheological properties and other properties that make it suitable for some medical applications. See Klock, G. et al., “Biocompatibility of mannuronic acid-rich alginates,” Biomaterials, Vol. 18, No. 10, 707-13 (1997). The use of alginate as a thickener for topical ophthalmic use is disclosed in U.S. Pat. No. 6,528,465 and U.S. Patent Application Publication 2003/0232089. In U.S. Pat. No. 5,776,445, alginate is used as a drug delivery agent that is topically applied to the eye. U.S. Patent Publication No. 2003/0232089 teaches a dry-eye formulation that contains two polymer ingredients including alginate.

The alginate used in the compositions will typically have a number average molecular weight from about 20 kDa to 2000 kDa, or from about 100 kDa to about 1000 kDa, for example about 325 kDa. The concentration of alginate is from about 0.01 wt. % to about 2.0 wt. %. More, typically, the concentration of alginate is a from about 0.1 wt. % to about 0.5 wt. %.

Guar gum or any derivative such as the hydroxypropyl or hydroxypropyl trimonium chloride derivatives can also be used as a natural polymer Guar and its derivatives are described in U.S. Pat. No. 6,316,506. Hydroxypropyl guar is of particular interest as a natural polymer for the described compositions. Guar gum and many of its derivatives are commercially available from Rhone-Poulenc (Cranbury, N.J.), Hercules, Inc. (Wilmington, Del.) and TIC Gum, Inc. (Belcamp, Md.). A preferred derivative for use in the compositions is hydroxypropyl guar.

Arabinogalactan is a wood sugars extracted from the Western Larch tree (also known as larch gum). Arabinogalactans are complex, highly branched polymers of arabinose and galactose in the ratio of from about 1:3 to about 1:10. A particular form of arabinogalactan is commercially available as Laracare®200 from Lonza, Inc.

Still another natural polymer is a fructan or any one fructan derivative. As used herein, the term “fructan” is understood to include all oligosaccharides and polysaccharides that have a majority of anhydrofructose units. The fructan can have a polydisperse chain length distribution and can be straight-chain or branched. The fructans include primarily β-2,6 bonds as in levan, or β-2,1 bonds as in inulin. One particular fructan derivative is carboxyl-modified fructan, e.g., inulin.

In many embodiments, the carboxyl-modified fructan includes 0.3 to 3 carboxyl groups per anhydrofructose unit. In particular, the carboxyl-modified fructan includes at least 0.8 carboxyl groups per anhydrofructose unit, e.g., from 1 to 2.2 carboxyl groups per anhydrofructose unit. The carboxyl groups can be present in the form of carboxyalkyl groups, for example, but not limited to, carboxymethyl, carboxyethyl, dicarboxymethyl or carboxyethoxycarbonyl groups. The carboxyl-modified fructans can be obtained by etherification of the fructan using synthetic methods well known in the art. Moreover, the carboxyl groups can also be present in the form of oxidized hydroxymethylene or hydroxymethyl groups. Any one mixture of different carboxyl-modified fructans can also be used. Also, the carboxyl-modified fructan can be a mixed carboxyl derivative, which can be prepared by etherfication of the fructan to a carboxymethylated form. The carboxymethylated form is then oxidized. The reverse reaction sequence is also possible. Carboxymethylinulin (CMI) is one of the more preferred carboxyl-modified fructans.

Carboxymethylinulin (CMI) with a DS (degree of substitution) of 0.15-2.5 is disclosed in WO 95/15984 and in the article by Verraest et al. in JAOCS, 73 (1996) pp. 55-62. As described, CMI can be prepared by the reaction of a concentrated solution of inulin with sodium chloroacetate at an elevated temperature. Carboxylethylinulin (CEI) is disclosed in WO 96/34017. The oxidation of inulin is disclosed in WO 91/17189 and WO 95/12619 (C₃-C₄ oxidation, leading to dicarboxylnulin, DCI) and WO 95/07303 (C6 oxidation).

The carboxyl-modified fructan has an average chain length (degree of polymerisation, DP) of at least 3, that is from 3 to 1000 monosaccharide units. More likely, the average chain length is from 6 to 60 monosaccharide units.

In some instances, one can prepare the carboxyl-modified fructan by first modifying the fructan itself. For example, the fructan can have its chain length enzymatically extended prior to carboxylation. Alternatively, the fructan can have its chain length shortened through a hydrolysis reaction. Fructans of a select chain length range can then be isolated by fractionation. Fractionation of fructans such as inulin can be effected by, for example, low temperature crystallisation (see WO 94/01849), column chromatography (see WO 94/12541), membrane filtration (see EP-A 440 074 and EP-A 627 490) or selective precipitation with alcohol. Hydrolysis to produce shorter fructans can, for example, be effected enzymatically (endo-inulinase), chemically (water and acid) or by heterogeneous catalysis (acid column).

Other types of anionic biopolymers that can be used in the compositions include carboxymethylcellulose and salts thereof, salts of carboxymethyl and carboxymethylhydroxyethyl starchs, and other glucoaminoglycans such as chondroitin sulfate, dermatan sulfate, heparin and heparin sulfate and keratin sulfates.

It is to be understood by those in the art that the compositions can include one or more of the natural polymers described above.

For applications as a multipurpose contact lens solution the compositions may require one or more cationic, antimicrobial components in addition to the one or more terpene compounds. Suitable cationic antimicrobial compounds include, but are not limited to, quaternary ammonium salts used in ophthalmic applications such as α-[4-tris(2-hydroxyethyl)ammonium chloride-2-butenyl]poly[1-dimethylammonium chloride-2-butenyl]-ω-tris(2-hydroxyethyl) ammonium chloride (hereafter, polyquaternium-1), benzalkonium halides, and biguanides such as salts of alexidine, alexidine-free base, salts of chlorhexidine, hexamethylene biguanides and salts thereof and poly(hexamethylenebiguanide) (hereafter, PHMB), antimicrobial polypeptides and any one mixture thereof.

In one embodiment, the antimicrobial component present in the ophthalmic compositions is a polymeric hexamethylene biguanide, which is present from 0.1 ppm to 0.8 ppm. In another embodiment, the primary antimicrobial component present in the lens care compositions is polyquaternium-1, which is present from 1 ppm to 5 ppm.

The ophthalmic compositions will very likely include a buffer system. A “buffer system” includes at least two buffer components that in combination provide the requisite buffering capacity, that is, the capacity to neutralize, within limits, either acids or bases (alkali) with relatively little or no change in the original pH. Generally, the buffering components are present from 0.05% to 2.5% (w/v) or from 0.1% to 1.5% (w/v).

The term “buffering capacity” is defined to mean the millimoles (mM) of strong acid or base (or respectively, hydrogen or hydroxide ions) required to change the pH by one unit when added to one liter (a standard unit) of the buffer solution. The buffer capacity will depend on the type and concentration of the buffer components. The buffer capacity is measured from a starting pH of 6 to 8, preferably from 7.4 to 8.4.

Borate buffer components include, for example, boric acid and its salts, for example, sodium borate or potassium borate. Borate buffer components also include compounds such as potassium tetraborate or potassium metaborate that produce borate acid or its salt in solutions. Borate buffers are known for enhancing the efficacy of certain polymeric biguanides. For example, U.S. Pat. No. 4,758,595 to Ogunbiyi et al. describes that a contact-lens solution containing PHMB can exhibit enhanced efficacy if combined with a borate buffer.

Phosphate buffer components include one or more monobasic phosphates, dibasic phosphates and the like. Particularly useful phosphate buffer components are those selected from phosphate salts of alkali and/or alkaline earth metals. Examples of suitable phosphate buffer components include one or more of sodium dibasic phosphate (Na₂HPO₄), sodium monobasic phosphate (NaH₂PO₄) and potassium monobasic phosphate (KH₂PO₄). The phosphate buffer components frequently are used in amounts from 0.01% or to 0.5% (w/v), calculated as phosphate ion.

Citrate buffer components include citric acid and any one of its salts, e.g. sodium citrate.

Other known buffer components can optionally be added to the lens care compositions, for example, sodium bicarbonate, TRIS, and the like. Other ingredients in the solution, while having other functions, may also affect the buffer capacity, e.g., propylene glycol or glycerin.

A preferred buffer system is one that includes a borate buffer component, a phosphate buffer component and a citrate buffer component. For example a combined boric/phosphate/citrate buffer system can be formulated from a mixture of boric acid/sodium borate, a monobasic/dibasic phosphate and citric acid/sodium citrate. In one embodiment, the buffer system comprises boric acid, a dibasic phosphate salt and sodium citrate.

Ophthalmic compositions formulated as a lens care solution will also include one or more surfactants. The surfactants can be cationic, amphoteric or nonionic, and are typically present (individually or in combination) in amounts up to 2% w/v. One preferred surfactant class are the nonionic surfactants. The surfactant should be soluble in the lens care solution and non-irritating to eye tissues. Many nonionic surfactants comprise one or more chains or polymeric components having oxyalkylene (—O—R—) repeats units wherein R has 2 to 6 carbon atoms. Preferred non-ionic surfactants comprise block polymers of two or more different kinds of oxyalkylene repeat units, which ratio of different repeat units determines the HLB of the surfactant. Satisfactory non-ionic surfactants include polyethylene glycol esters of fatty acids, e.g. coconut, polysorbate, polyoxyethylene or polyoxypropylene ethers of higher alkanes (C₁₂-C₁₈). Examples of this class include polysorbate 20 (available under the trademark Tween® 20), polyoxyethylene (23) lauryl ether (Brij® 35), polyoxyethyene (40) stearate (Myrj®52), polyoxyethylene (25) propylene glycol stearate (Atlas® G 2612). Still another preferred surfactant is tyloxapol.

A particular non-ionic surfactant consisting of a poly(oxypropylene)-poly(oxyethylene) adduct of ethylene diamine having a molecular weight from about 6,000 to about 24,000 daltons wherein at least 40 weight percent of said adduct is poly(oxyethylene) has been found to be particularly advantageous for use in cleaning and conditioning both soft and hard contact lenses. The CTFA Cosmetic Ingredient Dictionary's adopted name for this group of surfactants is poloxamine. Such surfactants are available from BASF Wyandotte Corp., Wyandotte, Mich., under Tetronic®. Particularly good results are obtained with poloxamine 1107 or poloxamine 1304. The foregoing poly(oxyethylene) poly(oxypropylene) block polymer surfactants will generally be present in a total amount from 0.0 to 2% w/v, from 0. to 1% w/v, or from 0.2 to 0.8% w/v

An analogous of series of surfactants, for use in the lens care compositions, is the poloxamer series which is a poly(oxyethylene) poly(oxypropylene) block polymers available under Pluronic® (commercially available form BASF). In accordance with one embodiment of a lens care composition the poly(oxyethylene)-poly(oxypropylene) block copolymers will have molecular weights from 2500 to 13,000 daltons or from 6000 to about 12,000 daltons. Specific examples of surfactants which are satisfactory include: poloxamer 108, poloxamer 188, poloxamer 237, poloxamer 238, poloxamer 288 and poloxamer 407. Particularly good results are obtained with poloxamer 237 or poloxamer 407. The foregoing poly(oxyethylene) poly(oxypropylene) block polymer surfactants will generally be present in a total amount from 0.0 to 2% w/v, from 0. to 1% w/v, or from 0.2 to 0.8% w/v.

Another class of surfactants are the amphoteric surfactants. Suitable amphoteric surfactants include betaine and sulphobetaine surfactants, and derivatives thereof. The betaine or sulphobetaine surfactants are believed to contribute to the disinfecting properties of the compositions by increasing the permeability of the bacterial cell wall, thus allowing the terpene or other antimicrobial agents to enter the cell.

The amphoteric surfactants of general formula I are surface-active compounds with both acidic and alkaline properties. The amphoteric surfactants of general formula I include a class of compounds known as betaines. The betaines are characterized by a fully quaternized nitrogen atom and do not exhibit anionic properties in alkaline solutions, which means that betaines are present only as zwitterions at near neutral pH.

All betaines are characterized by a fully quaternized nitrogen. In alkyl betaines, one of the alkyl groups of the quaternized nitrogen is an alkyl chain with eight to thirty carbon atoms. One class of betaines is the sulfobetaines or hydroxysulfobetaines in which the carboxylic group of alkyl betaine is replaced by sulfonate. In hydroxysulfobetaines a hydroxy-group is positioned on one of the alkylene carbons that extend from the quaternized nitrogen to the sulfonate. In alkylamido betaines, an amide group is inserted as a link between the hydrophobic C₈-C₃₀alkyl chain and the quaternized nitrogen.

The betaines are defined by general formula I

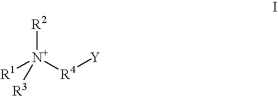

wherein R¹ is R or —(CH₂)ₙ—NHC(O)R, wherein R is a C₈-C₂₄alkyl optionally substituted with hydroxyl and n is 2, 3 or 4; R² and R³ are each independently selected from the group consisting of hydrogen and C₁-C₄alkyl; R⁴ is a C₂-C₆alkylene optionally substituted with hydroxyl; and Y is CO₂⁻ or SO₃⁻.

A sulfobetaine is defined by general formula II

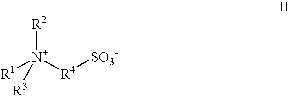

wherein R¹ is a C₈-C₁₆alkyl; R² and R³ are each independently selected from a C₁-C₄alkyl; and R⁴ is a C₂-C₆alkylene.

Certain sulfobetaines of general formula II are more preferred than others. For example, Zwitergent®3-10 available from Calbiochem Company, is a sulfobetaine of general formula I wherein R¹ is a straight, saturated alkyl with ten (10) carbons, R² and R³ are each methyl and R⁴ is —CH₂CH₂CH₂— (three carbons, (3)). Other sulfobetaines that can be used in the ophthalmic compositions include the corresponding Zwitergent®3-08 (R is a is a straight, saturated alkyl with eight carbons), Zwitergent®3-12 (R¹ is a is a straight, saturated alkyl with twelve carbons), Zwitergent®3-14 (R¹ is a is a straight, saturated alkyl with fourteen carbons) and Zwitergent®3-16 (R¹ is a is a straight, saturated alkyl with sixteen carbons). Of those mentioned, Zwitergent®3-10 is the most preferred because of the observed formulation properties such as solution compatibility, which is very important for a lens care solution.

A hydroxysulfobetaine is defined by general formula III

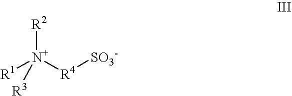

wherein R¹ is a C₈-C₁₆alkyl substituted with at least one hydroxyl; R² and R³ are each independently selected from a C₁-C₄alkyl; and R⁴ is a C₂-C₆alkylene substituted with at least one hydroxyl. An alkylamido betaine is defined by general formula IV

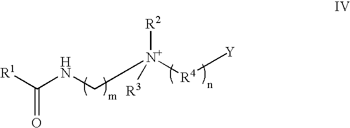

wherein R¹ is a C₈-C₁₆alkyl, and m and n are independently selected from 2, 3, 4 or 5; R² and R³ are each independently selected from a C₁-C₄alkyl optionally substituted with hydroxyl; R⁴ is a C₂-C₆alkylene optionally substituted with hydroxyl; and Y is CO₂⁻ or SO₃⁻. The most common alkylamido betaines are alkylamidopropyl betaines, e.g., cocoamidopropyl dimethyl betaine and lauroyl amidopropyl dimethyl betaine.

The ophthalmic compositions can also include one or more neutral or basic amino acids. The neutral amino acids include: the alkyl-group-containing amino acids such as alanine, isoleucine, valine, leucine and proline; hydroxyl-group-containing amino acids such as serine, threonine and 4-hydroxyproline; thio-group-containing amino acids such as cysteine, methionine and asparagine. Examples of the basic amino acid include lysine, histidine and arginine. The one or more neutral or basic amino acids are present in the compositions at a total concentration of from 0.1% to 5% (w/v).

The ophthalmic compositions can also include glycolic acid, aspartic acid or any mixture of the two at a total concentration of from 0.001% to 4% (w/v) or from 0.01% to 2.0% (w/v).

The ophthalmic compositions can also include diglycine. The amount of diglycine or salts thereof in the composition is from 0.01 wt. % to 2 wt. %, 0.05 wt. % to 2 wt. %, 0.1 wt. % to 2 wt. % or from 0.1 wt. % to 0.5 wt. %.

The ophthalmic compositions can also include dexpanthenol, which is an alcohol of pantothenic acid, also called Provitamin B5, D-pantothenyl alcohol or D-panthenol. In some formulations of the lens care compositions, dexpanthenol can exhibit good cleansing action and can stabilize the lachrymal film at the eye surface when placing a contact lens on the eye. Dexpanthenol is preferably present in the contact lens care compositions in an amount from 0.2% to 3% (w/v) or from 0.5% to 1.0% (w/v).

The ophthalmic compositions can also include a sugar alcohol, e.g., sorbitol, which is a hexavalent sugar alcohol, or xylitol, which is a pentavalent sugar. The sugar alcohol is present in the ophthalmic compositions in an amount from 0.1% to 1.0% (w/v), or from 0.2% to 0.5% (w/v).

In some embodiments, dexpanthenol is used with the sugar alcohol because the combination can provide enhanced cleansing action and can stabilize the lachrymal film following placement of the contact lens on the eye. These formulations can substantially improve patient comfort when wearing contact lenses.

The ophthalmic compositions can also include allantoin. Allantoin has been used as a moisturizing or soothing component in dermatological, cosmetic and veterinary formulations. Allantoin is also believed to promote cell-proliferation, which can stimulate healthy tissue formation. Allantoin is effective at quite low concentrations, 0.05% to 1% by weight.

Yet another suitable ophthalmic composition component comprises one or more viscosity control/wetting agents. Because of the demulcent effect of viscosity control and wetting agents, these materials have a tendency to enhance a contact lens wearer's comfort by means of a film on the lens surface cushioning impact against the eye. Suitable viscosity control/wetting agents include, for example, but are not limited to cellulose polymers like hydroxyethylcellulose or hydroxypropylcellulose, polyquaternium-10, and carboxymethylcellulose; povidone; poly(vinyl alcohol), poly(ethylene oxide) and poly(N,N-dimethylacrylamide) and the like. Viscosity control and wetting agents of the foregoing types are also described in greater detail in PCT Patent Application Nos. WO 04/093545 and WO 05/053759, both of which are again incorporated herein by reference. Viscosity control/wetting agents may be employed in the ophthalmic compositions in amounts ranging from about 0.001 wt % to about 1.0 wt % or less.

One exemplary ophthalmic composition is formulated as a contact lens disinfecting solution prepared with the components and amounts of each listed in Tables 1 to 4.

TABLE 1

| | Minimum | | Maximum | | Preferred | |
|---|---|---|---|---|---|---|
| | Amount | | Amount | | Amount | |
| Component | (wt. %) | | (wt. %) | | (wt. %) | |
| boric acid | 0.10 | | 1.0 | | 0.64 | |
| sodium borate | 0.01 | | 0.20 | | 0.1 | |
| sodium chloride | 0.20 | | 0.80 | | 0.49 | |
| zwitergent ® 3-10 | 0.005 | | 0.80 | | 0.1 | |
| hyaluronic acid | 0.005 | | 0.2 | | 0.02 | |
| 1-terpene-4-ol | 0.4 | ppm | 1.8 | ppm | 4.0 | ppm |
| Tetronic ® 1107 | 0.05 | | 2.0 | | 1.00 | |
| Na₂EDTA | 0.005 | | 0.15 | | 0.03 | |

TABLE 2

| | Minimum | | Maximum | | Preferred | |
|---|---|---|---|---|---|---|
| | Amount | | Amount | | Amount | |
| Component | (wt. %) | | (wt. %) | | (wt. %) | |
| sorbitol or xylitol | 0.5 | | 5 | | 3 | |
| poloxamer 407 | 0.05 | | 1.0 | | 0.10 | |
| Na diphosphate, | 0.10 | | 0.8 | | 0.46 | |
| dexpanthenol | 0.01 | | 1.0 | | 0.03 | |
| zwitergent ® 3-10 | 0.01 | | 0.2 | | 0.05 | |
| arabinogalactan | 0.05 | | 0.4 | | 0.1 | |
| 1-terpene-4-ol | 0.4 | ppm | 1.8 | ppm | 4.0 | ppm |
| allantoin | 0.05 | | 0.25 | | 1.0 | |
| Na₂EDTA | 0.005 | | 0.3 | | 0.1 | |

TABLE 3

| | Minimum | | Maximum | | Preferred | |
|---|---|---|---|---|---|---|
| | Amount | | Amount | | Amount | |
| Component | (wt. %) | | (wt. %) | | (wt. %) | |
| propylene glycol | 0.1 | | 1.0 | | 0.50 | |
| poloxamer 237 | 0.01 | | 0.20 | | 0.05 | |
| Na monophosphate | 0.05 | | 0.40 | | 0.10 | |
| Na diphosphate | 0.05 | | 0.4 | | 0.12 | |
| zwitergent ® 3-10 | 0.01 | | 0.3 | | 0.1 | |
| alginate | 0.005 | | 0.2 | | 0.02 | |
| 1-terpene-4-ol | 0.4 | ppm | 1.8 | ppm | 4.0 | ppm |
| allantoin | 0.05 | | 0.25 | | 1.0 | |
| Na₂EDTA | 0.005 | | 0.3 | | 0.1 | |

TABLE 4

| | Minimum | | Maximum | | Preferred | |
|---|---|---|---|---|---|---|
| | Amount | | Amount | | Amount | |
| Component | (wt. %) | | (wt. %) | | (wt. %) | |
| propylene glycol | 0.2 | | 2.0 | | 0.6 | |
| poloxamine 1304 | 0.01 | | 0.2 | | 0.05 | |
| boric acid | 0.1 | | 1.0 | | 0.60 | |
| Na borate | 0.01 | | 0.2 | | 0.10 | |
| hydroxypropyl guar | 0.01 | | 0.4 | | 0.05 | |
| 1-terpene-4-ol | 0.4 | ppm | 1.8 | ppm | 4.0 | ppm |
| allantoin | 0.05 | | 0.25 | | 1.0 | |
| zwitergent ® 3-10 | 0.01 | | 0.2 | | 0.05 | |
| Na₂EDTA | 0.02 | | 0.1 | | 0.05 | |

Yet another suitable ophthalmic composition component comprises one or more tonicity agents. Tonicity agents (also called osmolality-adjusting agents) serve to have the compositions herein approximate the osmotic pressure of normal lachrymal fluids, which is equivalent to a 0.9 percent solution of sodium chloride or 2.5 percent glycerin solution. Examples of suitable tonicity agents include but are not limited to sodium and potassium chloride; monosaccharides such as dextrose, mannose, sorbitol and mannitol; low molecular weight polyols such as glycerin and propylene glycol; and calcium and magnesium chloride. These tonicity agents are typically used individually in the ophthalmic compositions herein in amounts ranging from about 0.01 wt % to about 2.5 percent wt %.

The compositions can be used as a disinfecting solution, a preservative solution or packaging solution for contact lenses including (1) hard lenses formed from materials prepared by polymerization of acrylic esters such as polymethyl methacrylate (PMMA), (2) rigid gas permeable (RGP) lenses formed from silicone acrylates and fluorosilicone methacrylates, (3) soft, hydrogel lenses, and (4) non-hydrogel elastomer lenses.

As an example, soft hydrogel contact lenses are made of a hydrogel polymeric material, a hydrogel being defined as a crosslinked polymeric system containing water in an equilibrium state. In general, hydrogels exhibit excellent biocompatibility properties, i.e., the property of being biologically or biochemically compatible by not producing a toxic, injurious or immunological response in a living tissue. Representative conventional hydrogel contact lens materials are made by polymerizing a monomer mixture comprising at least one hydrophilic monomer, such as (meth)acrylic acid, 2-hydroxyethyl methacrylate (HEMA), glyceryl methacrylate, N,N-dimethacrylamide, and N-vinylpyrrolidone (NVP). In the case of silicone hydrogels, the monomer mixture from which the copolymer is prepared further includes a siloxy-containing monomer, in addition to the hydrophilic monomer. Generally, the monomer mixture will include a crosslinking monomer, i.e., a monomer having at least two polymerizable radicals, such as ethylene glycol dimethacrylate, tetraethylene glycol dimethacrylate, and methacryloxyethyl vinylcarbonate. Alternatively, either the siloxy-containing monomer or the hydrophilic monomer may function as a crosslinking agent.

The invention relates is also directed to a method of treating a patient with dry eye using the aqueous ophthalmic compositions described herein. The method includes administering the ophthalmic composition to the eye, eye lid or to the skin surrounding the eye. The compositions are thus useful for relieving eye irritation or dryness and providing lubrication for the eyes, irrespective of whether contact lenses are present in the eyes of the patient.

The ophthalmic compositions can be formulated to function as artificial tears and can be used, as needed, for the temporary relief of eye irritation or discomfort. For example, many people suffer from temporary or chronic eye conditions in which the eye's tear system fails to provide adequate tear volume or tear film stability necessary to remove irritating environmental contaminants such as dust, pollen, or the like. In persons suffering from chronic dry eye, the film on the eye tends to becomes discontinuous. The ophthalmic compositions can be used to treat the above conditions.

The invention is also directed to a method for preserving, disinfecting or cleaning contact lenses. In general, such a method comprises contacting the lenses with an ophthalmic composition. Although such contacting may be accomplished by simply soaking a lens in the ophthalmic composition, greater preserving, disinfecting or cleaning may possibly be achieved if a few drops of the composition are initially placed on each side of the lens, and the lens is rubbed for a period of time, for example, approximately 20 seconds. The lens can then be subsequently immersed within several milliliters of the subject composition. Preferably, the lens is permitted to soak in the composition for at least four hours. Furthermore, the lens is preferably rinsed with fresh composition after any rubbing step and again after being immersed within the composition. The lenses can then be removed from the composition, rinsed with the same or a different liquid, for example, a preserved isotonic saline solution and placed on the eye.

The ophthalmic compositions are illustrated by the following examples described in Tables 5 to 8. Each component is listed in w/v % unless noted in ppm.

In the examples below, certain chemical ingredients are identified by the following abbreviations.

EDTA: EthylenediamineTetraacetic Acid

PHMB: Poly(hexamethylene biguanide)

Dequest®2016 Tetrasodium phosphate, (1-hydroxyethylidene)diphosphonic acid, sodium salt, available from Monsanto Co. (Hydroxyalkylphosphonate (30%))

Tetronic® 1107: a surfactant, commercially available from BASF.

Pluronic® P123: a surfactant, commercially available from BASF.

Pluronic® F127: a surfactant, commercially available from BASF.

TABLE 5

| | Example | | |
|---|---|---|---|
| | 1 | 2 | 3 |
| boric acid | 0.85 | 0.85 | 0.85 |
| Na monophosphate | 0.31 | 0.31 | 0.31 |
| Na diphosphate | 0.15 | 0.15 | 0.15 |
| Dequest ® 2016 | 0.03 | 0.03 | 0.03 |
| Tetronic ® 1107 | 1.00 | 1.00 | 1.00 |
| geraniol (ppm) | 1.0 | — | — |
| α-terpineol (ppm) | — | 1.0 | — |
| 1-terpene-4-ol | — | — | 1.0 |
| (ppm) | | | |
| hyaluronic acid | 0.05 | 0.05 | 0.05 |
| (ppm) | | | |
| purified water | Q.S. | Q.S. | Q.S. |
| | to 100% | to 100% | to 100% |

TABLE 6

| | Example | | | |
|---|---|---|---|---|
| | Comp. | | Comp. | 5 |
| | Ex. 1 | 4 | Ex. 2 | % (w/w) |
| boric acid | 0.85 | 0.85 | 0.85 | 0.85 |
| Na monophosphate | 0.15 | 0.15 | 0.15 | 0.15 |
| Na diphosphate | 0.31 | 0.31 | 0.31 | 0.31 |
| NaCl | 0.19 | 0.19 | 0.19 | 0.19 |
| Dequest ® 2016 | 0.03 | 0.03 | 0.03 | 0.03 |
| Tetronic ® 1107 | 1.00 | 1.00 | 1.00 | 1.00 |
| alginate | — | 0.1 | — | 0.1 |
| 1-terpene-4-ol (ppm) | 1.5 | 1.5 | — | — |
| geraniol (ppm) | — | — | 1.5 | 1.5 |
| purified water | Q.S. | Q.S. | Q.S. | Q.S. |
| | to 100% | to 100% | to 100% | to 100% |

TABLE 7

| | Example | | | |
|---|---|---|---|---|
| | Comp. | | Comp. | |
| | Ex. 3 | 6 | Ex. 4 | 7 |
| boric acid | 0.85 | 0.85 | 0.85 | 0.85 |
| Na monophosphate | 0.15 | 0.15 | 0.15 | 0.15 |
| Na diphosphate | 0.31 | 0.31 | 0.31 | 0.31 |
| NaCl | 0.19 | 0.19 | 0.19 | 0.19 |
| Dequest ® 2016 | 0.03 | 0.03 | 0.03 | 0.03 |
| Tetronic ® 1107 | 1.00 | 1.00 | 1.00 | 1.00 |
| arabinogalactan | — | 0.1 | — | 0.1 |
| 1-terpene-4-ol (ppm) | 1.5 | 1.5 | — | — |
| geraniol (ppm) | — | — | 1.5 | 1.5 |
| purified water | Q.S. | Q.S. | Q.S. | Q.S. |
| | to 100% | to 100% | to 100% | to 100% |

TABLE 8

| | Example | | |
|---|---|---|---|
| | 8 | 9 | 10 |
| boric acid | 0.85 | 0.85 | 0.85 |
| Na monophosphate | 0.31 | 0.31 | 0.31 |
| Na diphosphate | 0.15 | 0.15 | 0.15 |
| Dequest ® 2016 | 0.03 | 0.03 | 0.03 |
| Tetronic ® 1107 | 1.00 | 1.00 | 1.00 |
| allantoin | 0.25 | 0.25 | 0.35 |
| geraniol (ppm) | 1.0 | — | — |
| α-terpineol (ppm) | — | 1.0 | — |
| 1-terpene-4-ol | — | — | 1.0 |
| (ppm) | | | |
| hyaluronic acid | 0.05 | 0.05 | 0.05 |
| (ppm) | | | |
| purified water | Q.S. | Q.S. | Q.S. |
| | to 100% | to 100% | to 100% |

What is claimed is:

1. An aqueous ophthalmic composition comprising:
one or more natural polymers selected from the group comprising hyaluronic acid, condroitin sulfate, alginate, guar gum, fructan and arabinogalactan or any corresponding salt or derivative of each thereof, and
one or more terpene compounds, wherein the ophthalmic composition has an osmolality in a range from 200 mOsmol/kg to 400 mOsmol/kg.

2. The composition of claim 2 further comprising polymeric hexamethylene biguanide, which is present from 0.2 ppm to 0.8 ppm, α-[4-tris(2-hydroxyethyl) ammonium chloride-2-butenyl]poly[1-dimethylammonium chloride-2-butenyl]-ω-tris(2-hydroxyethyl) ammonium chloride, which is present from 1 ppm to 5 ppm, or any one mixture thereof.

3. The composition of claim 1 wherein the natural polymers are selected from the group consisting of hyaluronic acid, alginate and arabinogalactan.

4. The composition of claim 4 wherein the terpene compounds are selected from the group consisting of α-terpineol, 1-terpinen-4-ol, menthol, eugenol and geraniol.

5. The composition of claim 1 further comprising dexpanthenol, sorbitol, xylitol or any one mixture thereof.

6. The composition of claim 1 further comprising a compound of formula I 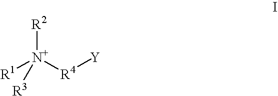
wherein R¹ is R or —(CH₂)ₙ—NHC(O)R, wherein R is a C₈-C₂₄alkyl optionally substituted with hydroxyl and n is 2, 3 or 4; R² and R³ are each independently selected from the group consisting of hydrogen and C₁-C₄alkyl; R⁴ is a C₂-C₆alkylene optionally substituted with hydroxyl; and Y is CO₂⁻ or SO₃⁻.

7. The composition of claim 6 wherein R¹ is decyl, and R² and R³ are methyl, and Z is —SO₃⁻.

8. The composition of claim 1 wherein the natural polymers are selected from hydroxypropyl guar or carboxyl-modified fructan.

9. The composition of claim 1 further comprising allantoin.

10. An aqueous ophthalmic composition comprising:
one or more natural polymers selected from the group comprising hyaluronic acid, hydroxypropyl guar and alginate or any corresponding salt or derivative of each thereof;
one or more terpene compounds; and
a sugar alcohol, wherein the ophthalmic composition has an osmolality in a range from 200 mOsmol/kg to 400 mOsmol/kg.

11. The composition of claim 10 further comprising a compound of formula I 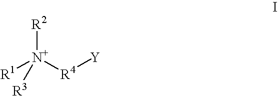
wherein R¹ is R or —(CH₂)ₙ—NHC(O)R, wherein R is a C₈-C₃₀alkyl optionally substituted with hydroxyl and n is 2, 3 or 4; R² and R³ are each independently selected from the group consisting of hydrogen and C₁-C₄alkyl; R⁴ is a C₂-C₈alkylene optionally substituted with hydroxyl; and Y is CO₂⁻ or SO₃⁻,

12. The composition of claim 10 wherein the natural polymers are selected from hyaluronic acid or alginate.

13. The composition of claim 10 wherein the natural polymers are selected from hydroxypropyl guar or carboxyl-modified fructan.

14. The composition of claim 10 wherein the terpene compounds are selected from the group consisting of α-terpineol, 1-terpinen-4-ol, menthol, eugenol and geraniol.

15. The composition of claim 10 wherein the sugar alcohol is selected from dexpanthenol, sorbitol, xylitol or any one mixture thereof.

16. The composition of claim 10 further comprising allantoin.

17. The use of the ophthalmic composition of claim 1 in an eye care or a contact lens care product selected from the group consisting of eye drops, contact lens packaging solution and contact lens multi-purpose solution.

18. The use of the ophthalmic composition of claim 10 in an eye care or a contact lens care product selected from the group consisting of eye drops, contact lens packaging solution and contact lens multi-purpose solution.
